(19) United States
(12) Patent Application Publication
(10) Pub. No.: US 2025/0213459 A1
(43) Pub. Date: Jul. 3, 2025

(54) METHOD OF CULTIVATING HIBISCUS HAMABO'S ADVENTITIOUS ROOT AND COSMETIC COMPOSITION COMPRISING THE EXTRACT OF HIBISCUS HAMABO'S ADVENTITIOUS ROOT CULTIVATED THEREBY

(71) Applicant: UCL CO., LTD., Jeju-si (KR)

(72) Inventors: Jong-Du Lee, Jeju (KR); Boram Go, Jeju-do (KR); Young-Min Ham, Jeju-do (KR); Sung Chun Kim, Jeju-do (KR); Seon-A Yoon, Jeju-do (KR); Hyejin Hyeon, Jeju-do (KR); Yu-Jung Han, Jeju-do (KR); Eun Bi Jang, Chungcheongbuk-do (KR); Ho Bong Hyun, Jeju-do (KR); Yong-Hwan Jung, Jeju-do (KR); Jeong Mi Kim, Jeju-do (KR); Ji Young Moon, Jeju-do (KR)

(21) Appl. No.: 19/002,106

(22) Filed: Dec. 26, 2024

(30) Foreign Application Priority Data
Dec. 27, 2023 (KR) 10-2023-0192980

(51) Int. Cl. A61K 8/9789; A61Q 19/00; C12N 5/00; C12N 5/04

(52) U.S. Cl. CPC A61K 8/9789; A61Q 19/00; C12N 5/0025; C12N 5/04

(57) ABSTRACT

The present disclosure provides a method for culturing Hibiscus hamabo's adventitious root, which includes a first culturing step of inoculating Hibiscus hamabo's adventitious root to a culture medium and culturing Hibiscus hamabo's adventitious root cells; and a second culturing step of sequentially treating the cultured Hibiscus hamabo's adventitious root with methyl jasmonate as an elicitor and culturing the same, and a method for preparing a Hibiscus hamabo's adventitious root using the same.

### CROSS-REFERENCE TO RELATED APPLICATION

This application claims priority of Korean Patent Application No. 10-2023-0192980, filed on Dec. 27, 2023, in the KIPO (Korean Intellectual Property Office), the disclosure of which is incorporated herein entirely by reference.

### BACKGROUND OF THE INVENTION

### Field of the Invention

The present disclosure relates to a technology for extraction of natural substances, specifically to a technology for culturing and extraction for enhancing the active ingredients of Hibiscus hamabo's adventitious root.

### Description of the Related Art

Hibiscus hamabo's adventitious root is the part that grows from the root of the plant Hibiscus hamabo. Hibiscus hamabo is a plant that belongs to the genus Hibiscus of the family Malvaceae. It is a deciduous shrub growing only in costal regions of Korea, Japan, Taiwan, etc. It is also a rare plant designated as endangered wildlife class II.

As a technology that utilizes Hibiscus hamabo, Korean Patent Registration No. 10-2018533 discloses a composition for skin antipollution contains containing a Hibiscus hamabo extract, a fraction of the Hibiscus hamabo extract, or a compound separated from the fraction of the Hibiscus hamabo extract, which is less irritant to skin and has superior antipollution effect of protecting the skin from environmental pollutants.

However, the above-described technology relates to simple extraction using a hydrophilic solvent such as water, ethanol, acetone, etc., and is limited in terms of the extraction efficiency of active ingredients and diversification of the efficacy of the extracted active ingredients.

In addition, since the type and functionality of active ingredients extracted from a plant may vary depending on the specificity of the part of the plant or extraction method, it is necessary to develop various methods for active and diversified applications of the rare Hibiscus hamabo.

### SUMMARY OF THE INVENTION

The present disclosure has been designed to utilize Hibiscus hamabo, which is a rare species, and enhance its active ingredients, and is directed to providing a method for culturing Hibiscus hamabo's adventitious root, which can significantly improve the extraction efficiency of the active ingredients in Hibiscus hamabo.

The present disclosure is also directed to providing a method for preparing an effective extract from cultured Hibiscus hamabo's adventitious root cells.

The present disclosure is also directed to providing a Hibiscus hamabo's adventitious root extract with a significantly increased content of active ingredients, obtained by the culturing and extraction methods described above.

The present disclosure is also directed to providing a cosmetic composition containing the Hibiscus hamabo's adventitious root extract, which contains at least 2.1 mg/g of N-[4′-hydroxy-cinnamoyl]-aspartic acid, and can increase the hyaluronic acid content in skin.

A method for culturing Hibiscus hamabo's adventitious root according to an exemplary embodiment of the present disclosure includes: a first culturing step of inoculating Hibiscus hamabo's adventitious root to a culture medium and culturing Hibiscus hamabo's adventitious root cells; and a second culturing step of sequentially treating the cultured Hibiscus hamabo's adventitious root with methyl jasmonate as an elicitor.

The culture medium may contain a MS (Murashige & Skog) salt, a plant growth regulator, and sugar.

The first culturing step may be conducted at 24 to 26° C., and the inoculation volume of the Hibiscus hamabo's adventitious root may be 3 to 5 g/L.

The plant regulator may include indole-3-butyric acid.

Specifically, the pH of the culture medium may be adjusted to 5.7 to 5.8.

It is advantageous in terms of the yield and content of active ingredients to supply the methyl jasmonate to the culture medium so that the concentration of the methyl jasmonate in the culture medium is maintained at 50 to 200 UM.

The method for preparing a Hibiscus hamabo's adventitious root extract of the present disclosure includes: a step of extracting the Hibiscus hamabo's adventitious root cells cultured by the method described above by immersing in an alcohol at room temperature and separating the Hibiscus hamabo's adventitious root extract; and a step of decompression-concentrating and freeze-drying the separated Hibiscus hamabo's adventitious root extract.

According to an exemplary embodiment, the Hibiscus hamabo's adventitious root extract is extracted by the method described above and has a content of N-[4′-hydroxy-cinnamoyl]-aspartic acid at least 2.1 mg/g.

According to an exemplary embodiment, the cosmetic composition is extracted by the method described above and has a content of N-[4′-hydroxy-cinnamoyl]-aspartic acid at least 2.1 mg/g.

In accordance with a method for culturing Hibiscus hamabo's adventitious root according to the present disclosure, it is possible not only to maximize the growth of Hibiscus hamabo's adventitious root cells, but also to dramatically increase the accumulation of active ingredients such as polyphenols, flavonoids, etc. within the cells. Therefore, it is possible to secure Hibiscus hamabo's adventitious root cells with enhanced active ingredients.

Furthermore, by providing an extraction condition that can maximize the active ingredients of Hibiscus hamabo's adventitious root, the present disclosure allows preparation of an extract having significantly increased contents of active ingredients from the grown Hibiscus hamabo's adventitious root cells as compared to the conventional technology.

It is expected that the extract obtained by the culturing and extraction methods described above can be developed into cosmetics with significantly improved skin-moisturizing and stabilizing properties as compared to the conventional cosmetics derived from natural products.

In commercial aspect, it will be possible to expand the application of the adventitious root of Hibiscus hamabo, which is a rare species, and increase the revenue of farmers.

### BRIEF DESCRIPTION OF THE DRAWINGS

The above and other features and advantages will become more apparent to those of ordinary skill in the art by describing in detail exemplary embodiments with reference to the attached drawings, in which:

FIG. 1 is a flowchart conceptually showing a method for preparing a Hibiscus hamabo's adventitious root extract according to an exemplary embodiment of the present disclosure.

FIG. 2 shows a result of HPLC chromatogram analysis of extracts obtained in examples and comparative examples.

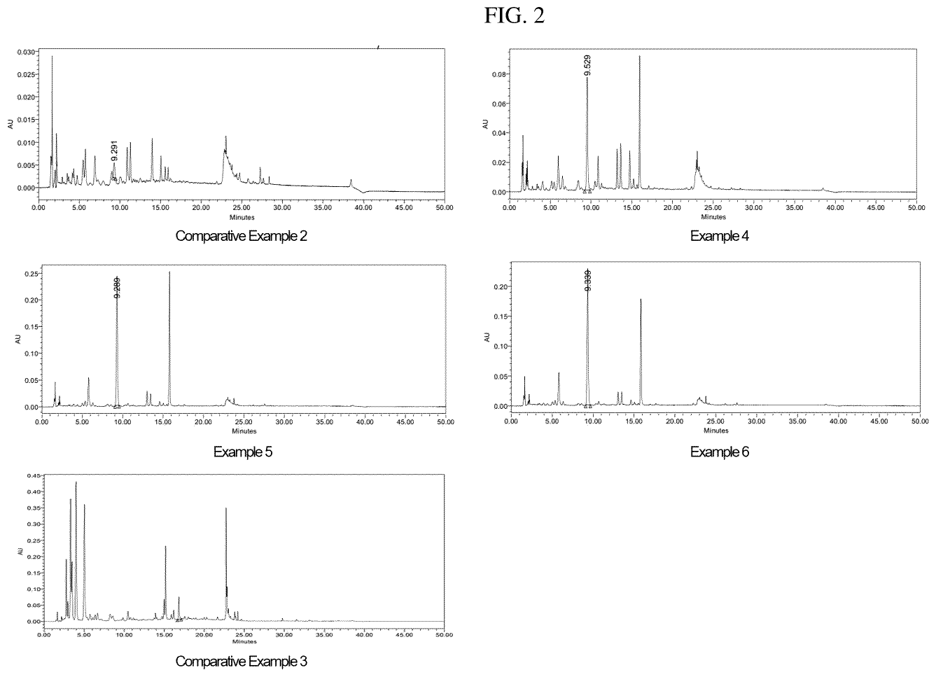

FIG. 3 shows a result of evaluating the cytotoxicity of extracts obtained in examples and comparative examples.

FIG. 4 shows a result of investigating hyaluronic acid synthesis in extracts obtained in examples and comparative examples.

In the following description, the same or similar elements are labeled with the same or similar reference numbers.

### DETAILED DESCRIPTION

The present invention now will be described more fully hereinafter with reference to the accompanying drawings, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “includes”, “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. In addition, a term such as a “unit”, a “module”, a “block” or like, when used in the specification, represents a unit that processes at least one function or operation, and the unit or the like may be implemented by hardware or software or a combination of hardware and software.

Reference herein to a layer formed “on” a substrate or other layer refers to a layer formed directly on top of the substrate or other layer or to an intermediate layer or intermediate layers formed on the substrate or other layer. It will also be understood by those skilled in the art that structures or shapes that are “adjacent” to other structures or shapes may have portions that overlap or are disposed below the adjacent features.

In this specification, the relative terms, such as “below”, “above”, “upper”, “lower”, “horizontal”, and “vertical”, may be used to describe the relationship of one component, layer, or region to another component, layer, or region, as shown in the accompanying drawings. It is to be understood that these terms are intended to encompass not only the directions indicated in the figures, but also the other directions of the elements.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Preferred embodiments will now be described more fully hereinafter with reference to the accompanying drawings. However, they may be embodied in different forms and should not be construed as limited to the embodiments set forth herein. Rather, these embodiments are provided so that this disclosure will be thorough and complete, and will fully convey the scope of the disclosure to those skilled in the art.

Hereinafter, a method for preparing a Hibiscus hamabo's adventitious root extract according to an exemplary embodiment of the present disclosure will be described in detail referring to the attached drawings. A method for culturing Hibiscus hamabo's adventitious root will also be described. Furthermore, the obtained Hibiscus hamabo's adventitious root extract and the utilization thereof will also be described. The following description is exemplary explanation for specifying and explaining the technical idea of the present disclosure, and the technical idea of the present disclosure is not limited by the following description. The technical idea of the present disclosure can only be interpreted and limited by the scope of the claims described below.

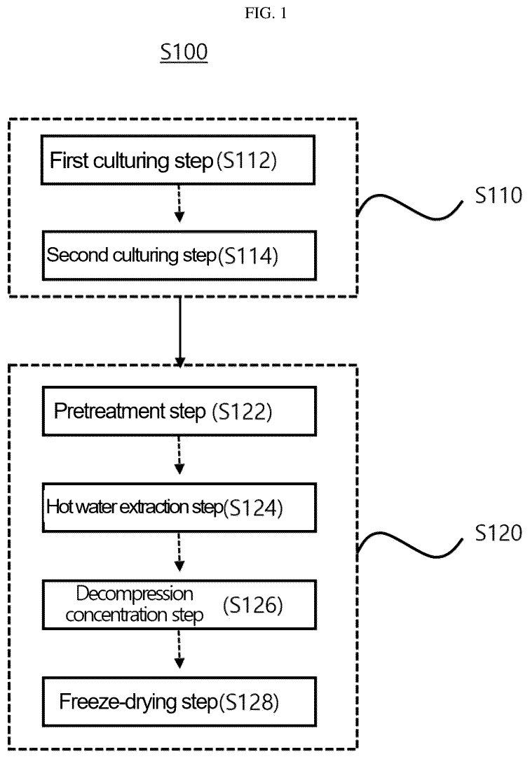

FIG. 1 is a flowchart conceptually showing a method for preparing a Hibiscus hamabo's adventitious root extract according to an exemplary embodiment of the present disclosure.

Referring to FIG. 1, a method for preparing a Hibiscus hamabo's adventitious root extract S100 includes a Hibiscus hamabo's adventitious root culturing step S110 and a Hibiscus hamabo's adventitious root extraction step S120. The Hibiscus hamabo's adventitious root extraction step S120 is a step in which a Hibiscus hamabo's adventitious root extract is substantially produced.

The Hibiscus hamabo's adventitious root culturing step S110 includes a first culturing step S112 and a second culturing step S11) as a continuous process. The first culturing step S112 is a step of inoculating Hibiscus hamabo's adventitious root to a culture medium and growing the same. In this exemplary embodiment, the culture medium contains a MS (Murashige & Skog) salt, a plant growth regulator, and sucrose.

The MS salt is a known culture medium developed in 1962 by American plant physiologists Murashige and Skoog. It is composed of minerals and organic materials and supplies nutrients necessary for plant growth.

As the plant growth regulator, indole-3-butyric acid (IBA), a-naphthaleneacetic acid (NAA), or kinetin may be used. In this exemplary embodiment, a combination of two or more of them may be used as the plant growth regulator. IBA has a chemical structure similar to that of indole-3-acetic acid (IAA) and is effective in promoting root growth in plants. It is mainly used to promote root formation. Although IBA is more stable than indole acetic acid, it decomposes easily when exposed to light. It inhibits plant growth at high concentrations, induces the production of endogenous ethylene, and can promote the maturity and aging of plant tissues or organs.

The sucrose is a type of sugar. It is a disaccharide composed of glucose and galactose. In this exemplary embodiment, by including the sucrose in the culture medium, cell wall formation is promoted and cell differentiation and growth are assisted in the Hibiscus hamabo's adventitious root.

The first culturing step S112 is performed after the Hibiscus hamabo's adventitious root is inoculated to the culture medium with the composition described above.

The first culturing step S112 can last for about 7 to 8 weeks, although not being limited thereto. The duration of the first culturing step S112 can be variably in consideration of the purposes of use of the Hibiscus hamabo's adventitious root cells and the characteristics of the Hibiscus hamabo's adventitious root cells.

After the first culturing step S112 is completed, the second culturing step S114 is performed continuously. The second culturing step S114 is a step of treating the grown Hibiscus hamabo's adventitious root cells with methyl jasmonate (MJ) and culturing them. The methyl jasmonate acts as an elicitor. In the second culturing step S114, the concentration of methyl jasmonate is important. The methyl jasmonate is supplied continuously to the culture medium so that the concentration of the methyl jasmonate in the culture medium is maintained at 50 to 200 μM, more specifically 70 to 150 μM. However, the essential technical idea of the present disclosure is not limited by the concentration range described above.

The second culturing step S114 lasts approximately 1 week following the first culturing step S112, but this period can also be changed.

The culturing step S110 takes place in an environment strictly controlled at 24 to 26° C., and the pH of the culture medium is specifically controlled at 5.7 to 5.8.

After the culturing step S110 is completed, a Hibiscus hamabo's adventitious root extraction step S120 of preparing an effective Hibiscus hamabo's adventitious root extract from the Hibiscus hamabo's adventitious root cells obtained in the culturing step S110 is performed.

The Hibiscus hamabo's adventitious root extraction step S120 includes a pretreatment step S122, an extraction step S124, a decompression concentration step S126, and a freeze-drying step S128.

First, a pretreatment step S122 wherein the Hibiscus hamabo's adventitious root cells obtained in the culturing step S110 is filtered with a wicker tray to separate the culture medium and moisture is removed sufficiently is performed. The degree of moisture removal (drying) can be determined appropriately in consideration of the usage pattern of the Hibiscus hamabo's adventitious root extract. It is obvious that various known additional pretreatment processes required for subsequent processing can be performed.

In the extraction step S12), the prepared Hibiscus hamabo's adventitious root cells are leached at room temperature by immersing in an alcohol. As the alcohol, 50% ethanol may be used. The extraction step can take approximately a day. After the extraction step S124 is completed, only the filtrate is taken from the leached sample using a suction filter, etc.

The obtained filtrate can be prepared into an extract through a decompression concentration step S126 and a freeze-drying step S128. The decompression concentration step S126 and the freeze-drying step S128 can be performed using various known methods and devices. Specifically, the decompression concentration step S126 may be performed at a temperature of 35 to 45° C.

As a result of NMR analysis, the Hibiscus hamabo's adventitious root extract obtained by the method described above was confirmed to contain N-[4′-hydroxy-cinnamoyl]-aspartic acid. The content of the N-[4′-hydroxy-cinnamoyl]-aspartic acid in the extract was found to be at least 2.1 mg/g.

Meanwhile, it was confirmed that the extract can increase the content of hyaluronic acid when treated to HaCat cells. Details will be explained in the examples and test examples described below.

Since it was confirmed that the Hibiscus hamabo's adventitious root extract obtained through the Hibiscus hamabo's adventitious root culturing step S110 and the Hibiscus hamabo's adventitious root extraction step S120 according to an exemplary embodiment of the present disclosure contains highly enhanced active ingredients, it is expected that its utility will be very great.

In the following, specific examples will be used to explain the culturing method and extraction method described above in more detail. Furthermore, the characteristics of the products obtained through the culturing and extraction methods will be examined in detail through analysis of various experiment results.

### EXAMPLES

### Example 1: Culturing of Hibiscus hamabo's Adventitious Root (1)

Hibiscus hamabo's adventitious root cells were cultured in two steps in a 5-L bioreactor to maximize growth and accumulation of active ingredients simultaneously.

The first step was performed as follows, although not being limited thereto. (1) 3.5 to 4.0 L of a culture medium containing of ½ MS (Murashige & Skog) salt, indole-3-butyric acid (IBA, 2.0 mg/L), and sugar (sucrose, 30 g/L) was used. (2) The pH of the culture medium was adjusted to 5.7 to 5.8. (3) The inoculation volume of the adventitious root was 4.0 g/L. (4) The air supply rate was 0.1 vvm. (5) The culturing temperature was 25±1° C. (6) The cells were harvested after 8 to 9 weeks of culturing.

In the two-step culturing for accumulation of active ingredients such as polyphenols, flavonoids, etc., methyl jasmonate (MJ) was treated to Hibiscus hamabo's adventitious root cells, which had been grown for 7 to 8 weeks in a continuous immersion bioreactor, as an elicitor. Methyl jasmonate was treated for 1 week, from 7 to 8 weeks after starting the culturing. It was supplied at a concentration of 50 μM.

### Example 2: Culturing of Hibiscus hamabo's Adventitious Root (2)

Hibiscus hamabo's adventitious root was cultured in the same manner as in Example 1, except that methyl jasmonate was treated at a concentration of 100 UM.

### Example 3: Culturing of Hibiscus hamabo's Adventitious Root (3)

Hibiscus hamabo's adventitious root was cultured in the same manner as in Example 1, except that methyl jasmonate was treated at a concentration of 200 μM.

### Comparative Example 1: Culturing of Hibiscus hamabo's Adventitious Root (4)

Hibiscus hamabo's adventitious root was cultured in the same manner as in Example 1, except that the treatment with methyl jasmonate was omitted.

The biomass indices of the Hibiscus hamabo's adventitious root cells cultured through the two steps were analyzed as follows. Fresh weight was measured after separating the harvested cells from the culture medium by filtering with a stainless-steel wicker tray and sufficiently removing moisture with a clean tissue. After drying the cells at 40° C. for 2 to 3 days and measuring weight, the percentage of dry matter was calculated from the percentage of the dry weight to the fresh weight. Also, the growth index and relative growth index of the Hibiscus hamabo's adventitious root were calculated using the equations described below.

### [Test Example 1] Analysis of Biomass Indices

The biomass indices of the Hibiscus hamabo's adventitious root cells cultured through the two steps were analyzed. Fresh weight was measured after separating the harvested cells from the culture medium by filtering with a stainless-steel wicker tray and sufficiently removing moisture with a clean tissue. After drying the cells at 40° C. for 2 days and measuring weight, the percentage of dry matter was calculated from the percentage of the dry weight to the fresh weight. Also, the growth index and relative growth index of the callus were calculated using Equation 1 and Equation 2.

Growth index=[(fresh weight of finally grown cells)−(fresh weight of initially inoculated cells)]/(fresh weight of initially inoculated cells) [Equation 1]

Relative growth index=[natural logarithm (fresh weight of finally grown cells)−natural logarithm (fresh weight of initially inoculated cells)]/(culturing period, 8 weeks) [Equation 2]

The growth indices of the Hibiscus hamabo's adventitious root cells obtained in Examples 1 to 3 and Comparative Example 1 depending on treatment with methyl jasmonate are shown in Table 1.

TABLE 1

| | | | | | Relative |
|---|---|---|---|---|---|
| | Fresh | Dry | Percentage of | Growth | growth |
| Sample | weight (g) | weight (g) | dry matter (%) | index | index |
| Comparative | 332.24 | 11.35 | 3.42 | 19.77 | 0.38 |
| Example 1 | | | | | |
| Example 1 | 423.69 | 15.89 | 3.75 | 25.48 | 0.41 |
| Example 2 | 411.49 | 15.32 | 3.72 | 24.72 | 0.41 |
| Example 3 | 359.24 | 11.80 | 3.28 | 21.45 | 0.39 |

### Example 4: Extraction of Hibiscus hamabo's Adventitious Root (1)

The Hibiscus hamabo's adventitious root cells obtained in Example 1 was dried and crushed, and the crushed material was immersed in 50% ethanol and leached at room temperature for 24 hours. Only the filtrate was taken from the sample using a suction filter. The filtrate was decompression-concentrated in an aqueous solution at 40° C. and then freeze-dried to obtain an extract.

### Example 5: Extraction of Hibiscus hamabo's Adventitious Root (2)

A Hibiscus hamabo's adventitious root extract was obtained in the same manner as in Example 4, except that the Hibiscus hamabo's adventitious root cells obtained in Example 2 were used.

### Example 6: Extraction of Hibiscus hamabo's Adventitious Root (3)

A Hibiscus hamabo's adventitious root extract was obtained in the same manner as in Example 4, except that the Hibiscus hamabo's adventitious root cells obtained in Example 3 were used.

### Comparative Example 2: Extraction of Hibiscus Hamabo's Adventitious Root (4)

A Hibiscus hamabo's adventitious root extract was obtained in the same manner as in Example 4, except that the Hibiscus hamabo's adventitious root cells obtained in Comparative Example 1 were used.

### Comparative Example 3: Extraction of Wild Hibiscus hamabo Leaf

Hibiscus hamabo leaves collected from the wild were dried and crushed. The crushed leaves were immersed in 50% ethanol and leached at room temperature for 24 hours. Only the filtrate was taken from the sample using a suction filter. The filtrate was decompression-concentrated in a 40° C. aqueous solution and then freeze-dried to obtain an extract.

### [Test Example 2] Analysis of Extraction Yield

The result of analyzing yield after the extraction according to the methods described in Examples 4 to 6, and Comparative Examples 2 and 3 is shown in Table 2.

TABLE 2

| | Extraction | Extract | |
|---|---|---|---|
| Sample | amount (g) | amount (g) | Yield (%) |
| Comparative Example 2 | 10.04 | 1.16 | 11.6 |
| Example 4 | 20.00 | 4.43 | 22.2 |
| Example 5 | 5.01 | 0.83 | 16.6 |
| Example 6 | 5.00 | 1.01 | 20.3 |
| Comparative Example 3 | 10.02 | 1.66 | 16.6 |

### [Test Example 3] Analysis of Content of N-[4′-Hydroxy-Cinnamoyl]-Aspartic Acid Depending on Concentration of Methyl Jasmonate

N-[4′-Hydroxy-cinnamoyl]-aspartic acid (Chemical Formula 1) (Biosynth, Cat NO. FH180385, purity 99%) was dissolved in 50% methanol to 10,000 ppm. Afterwards, it was diluted to 100, 50, 25, 12.5, and 6.25 ppm using 50% methanol to create a calibration curve, and then filtered using a 0.5-μm syringe filter. The extracts of Test Example 2 were precisely weighed, prepared to 10,000 ppm by adding 50% methanol, and sonicated for 5 minutes. After filtering with a 0.5-μm syringe filter, the extract was used for analysis. The content of N-[4′-hydroxy-cinnamoyl]-aspartic acid was analyzed using the high-performance liquid chromatography (HPLC) system e2695 (Waters Co., Milford, MA, USA) equipped with the Waters photodiode array (PDA) detector 2998 (Waters Co., Milford, MA, USA), and the Sunfire C18 column (4.6×150 mm, 5 μm; Waters Co.) was used for separation of components. The column temperature was maintained at 40° C., and the sample injection volume was 10 μL. Solvent A (0.1% trifluoroacetic acid in water) and solvent B (acetonitrile) were used as a mobile phase, and the flow rate was constant at 1 mL/min. The gradient condition of the mobile phase was as follows: 0 min, 90% A/10% B; 5 min, 90% A/10% B; 20 min, 70% A/30% B; 21 min, 30% A/70% B; 25 min, 30% A/70% B; 26 min, 10% A/90% B; 36 min, 10% A/90% B; 37 min, 90% A/10% B; 50 min, 90% A/10% B. The UV spectrum range used for observation was 200 to 600 nm, and the measurement was made at a wavelength of 320 nm.

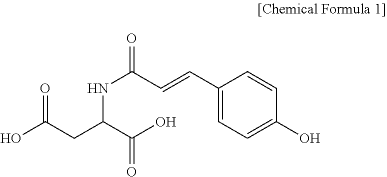 [SMILES: O=C(O)CC(NC(=O)/C=C/c1ccc(O)cc1)C(=O)O]

The result of the analysis of the N-[4′-hydroxy-cinnamoyl]-aspartic acid content is shown in Table 3.

TABLE 3

| | Content of N-[4′-hydroxy-cinnamoyl]- |
|---|---|
| Sample | aspartic acid (mg/g) |
| Comparative Example 2 | 0.07 |
| Example 4 | 2.1 |
| Example 5 | 6.5 |
| Example 6 | 6.2 |
| Comparative Example 3 | Not detected |

### [Test Example 4] Assessment of Cytotoxicity

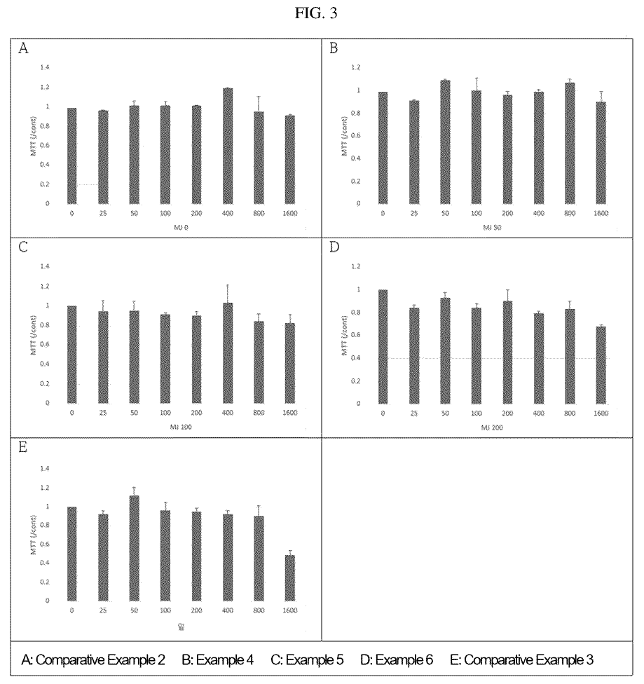

To measure cell viability, HaCaT cells were dispensed in a 24-well plate at 5×10⁵ cells/well and, after incubation for 24 hours, incubated with the sample at different concentrations for 24 hours. After treating the cultured cells with MTT solution at 0.5 mg/mL and conducting reaction at 37° C. for 2 hours, the resulting formazan was dissolved with DMSO and absorbance was measured at a wavelength of 570 nm using a microplate reader. Cell viability was calculated according to Equation 3 as a ratio with respect to the control group. FIG. 3 shows the result of evaluating the cytotoxicity of the extracts obtained in the examples and comparative examples.

Cell viability=absorbance of treatment group/absorbance of control group [Equation 3]

### [Test Example 5] Analysis of Hyaluronic Acid synthesis

Human keratinocytes (HaCaT) were seeded into a 24-well plate at 5×10⁴ cells/well. 24 hours later, the cells were treated with the sample at different concentrations after replacing the medium with a serum-free DMEM medium. After 24 hours of incubation, the cell culture was removed and centrifuged at 15,000 g for 5 minutes. The supernatant was removed to quantify hyaluronic acid using an R&D Quantikine hyaluronan ELISA kit. The change in hyaluronic acid synthesis was calculated according to Equation 4 as a ratio with respect to the control group.

Hyaluronic acid=absorbance of treatment group/absorbance of control group [Equation 4]

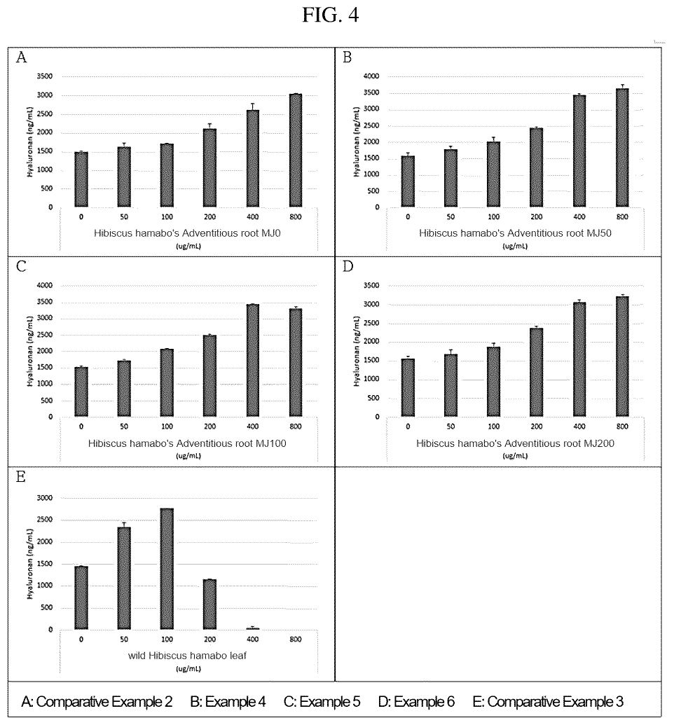

FIG. 4 shows a result of investigating hyaluronic acid synthesis in extracts obtained in examples and comparative examples.

As seen from the above experimental results, the content of N-[4′-hydroxy-cinnamoyl]-aspartic acid was increased by up to 92 times and the synthesis of hyaluronic acid was also increased by up to 1.4 times in the Hibiscus hamabo's adventitious root cells treated with methyl jasmonate at 50 to 200 μM, as compared to the group not treated with methyl jasmonate. As of 2022, Hibiscus hamabo is registered as endangered wildlife class II, and its biomass is very scarce for industrial application since it grows only in the southern area of Korea. It is expected that the method for culturing Hibiscus hamabo's adventitious root cells developed in the present disclosure, which provides increased N-[4′-hydroxy-cinnamoyl]-aspartic acid content and increased moisturizing effect, is industrially applicable through mass production.

What is claimed is:

1. A method of cultivating Hibiscus hamabo's adventitious root comprising:
a first culturing step of inoculating Hibiscus hamabo's adventitious root to a culture medium comprising a MS (Murashige & Skog) salt, indole-3-butyric acid as a plant regulator, and sucrose, and adjusted to pH 5.7 to 5.8 and culturing Hibiscus hamabo's adventitious root cells at a temperature of 24 to 26° C.;
a step of supplying methyl jasmonate to the cultured Hibiscus hamabo's adventitious root so that the concentration of methyl jasmonate is maintained at 50 to 200 μM;
a step of extracting cultured Hibiscus hamabo's adventitious root cells by immersing in an alcohol at room temperature and separating the Hibiscus hamabo's adventitious root extract; and
a step of decompression-concentrating and freeze-drying of the separated Hibiscus hamabo's adventitious root extract,
wherein the extract comprises at least 2.1 mg/g of N-[4′-hydroxy-cinnamoyl]-aspartic acid as an active ingredient.

2. The cosmetic composition according to claim 1, wherein the inoculation volume of the Hibiscus hamabo's adventitious root is 3 to 5 g/L.
